(19) United States
(12) Patent Application Publication
(10) Pub. No.: US 2011/0009961 A1
(43) Pub. Date: Jan. 13, 2011

(54) Radiopaque middle ear prosthesis

(75) Inventors: Paul Yarbrough, Bartlett, TN (US); Allen Palmer, Arlington, TN (US)

Correspondence Address: OLIFF & BERRIDGE, PLC, P.O. BOX 320850, ALEXANDRIA, VA, 22320-4850, US

(73) Assignee: GYRUS ENT, L.L.C., Bartlett, TN (US)

(21) Appl. No.: 12/458,462

(22) Filed: Jul. 13, 2009

(51) Int. Cl. A61F 2/18

(52) U.S. Cl. 623/10

(57) ABSTRACT

A biocompatible middle ear prosthesis includes a polymer and a radiopaque marking material. The biocompatible middle ear prosthesis is implanted in the middle ear to replace all or part of the middle ear ossicles. A method for detecting the location of biocompatible middle ear prosthesis including implanting at least one biocompatible middle ear prosthesis that includes a polymer and a radiopaque marking material into the middle ear, exposing the at least one biocompatible middle ear prosthesis to a radiation source, creating an image from the exposure of the biocompatible middle ear prosthesis to a radiation source, and viewing the image created from the exposure of the biocompatible middle ear prosthesis to a radiation source to determine the location of the biocompatible middle ear prosthesis in the middle ear.

### BACKGROUND

This disclosure relates to a biocompatible middle ear prosthesis with a radiopaque marking material incorporated into the biocompatible prosthesis. This disclosure also relates to a method for detecting the location of a biocompatible middle ear prosthesis with a radiopaque marking material by exposing the biocompatible middle ear prosthesis to a radiation source and viewing an image created by the exposure to the radiation source.

The mechanism for transmitting sonic vibrations from the tympanum to the inner ear functions via the ossicular chain. The stapes lies in the fenestra of the vestibule and provides communication between the middle ear and the inner ear. The mechanism transmits sonic vibrations through three articulated ossicles: the malleus, the incus, and the stapes. These three ossicles amplify the vibrations of the tympanum for transmitting sound to the inner ear.

FIG. 1 depicts the ossicles of the middle ear. The ear canal 10 funnels sound waves into the middle ear where the sound waves cause the tympanic membrane 20 to vibrate. The vibrations are transferred via the malleus 30 to the incus 40, and then transferred to the stapes 50. The stapes 50 contacts the oval window of the cochlea 60 where the vibrations are turned into amplified pressure waves in the fluid of the inner ear 70.

To optimally transmit sound from the outer ear to the inner ear it is required to have: effective contact between the malleus 30 and the tympanum 20 to sense sound induced vibrations of the tympanum 20; functional movement of the malleus 30, the incus 40 and the stapes 50 in response to the vibrations of the tympanum 20; and effective contact between the stapes 50 and the cochlea 60 to transmit sound vibrations to the inner ear 70.

Diseases of the middle ear, such as otosclerosis and congenital ear defects, can cause progressive hearing loss in adult life. Otosclerosis is the formation of spongy bone in the inner ear that causes the stapes to become immobilized. Early on, otosclerosis causes the nodule of the softened bone to become large enough to reach the oval window containing the footplate of the stapes. As the nodule increases in size, pressure impedes the vibratory movements of the stapes, and eventually the stapes becomes fixed.

Fixation of the stapes may be corrected by surgery. A common operation, a stapedectomy, involves removing the fixed stapes and replacing it with a plastic or wire prosthesis. The operation restores the vibratory characteristics of the stapes, thus restoring the function of the ossicles and allowing sound to be transmitted from the outer ear to the inner ear.

Middle ear prostheses are known in the art. For example, U.S. Pat. No. 3,191,188 discloses an artificial stapes consisting of a thin laminar base adaptable to the niche of the oval window from which lamina rises a tubular strut or stem that articulates with the incus, the strut being attached to the lenticular process of the incus thus replacing the mechanical link between the incus and the oval window. U.S. Pat. No. 3,196,462 discloses a prosthesis comprising an enlarged proximal portion that is hollow and open at the outer end for attachment to the incus. U.S. Pat. No. 3,473,170 discloses a prosthesis that attaches to the footplate of the stapes to replace the ossicles. U.S. Pat. No. 3,711,869 discloses a prosthesis that is attached to the under surface of the lenticular process of the incus. U.S. Pat. No. 6,197,060 discloses a prosthesis using a shape-memory metal alloy that self-secures about the incus when heat is applied to a pre-formed bight by means of a laser.

After a middle ear prosthesis is implanted, over a period of time, a patient's hearing may again deteriorate. One reason for the hearing deterioration is that the implanted prosthesis may become dislocated. However, other reasons, such as degeneration of other parts of the middle or inner ear, may be the cause of the hearing deterioration. Patients with middle ear prostheses occasionally undergo reevaluation due to persistent hearing loss or even vertigo and imbalance. A prosthesis displaced from the oval window may explain either conductive or sensorineural hearing loss. A prosthesis in contact and compressed against the saccule or utricle may explain vertigo. Therefore, there is a need for a physician to be able to view the location of the middle ear prosthesis in order to determine whether the location of the middle ear prosthesis has shifted. Currently, determining the location of a middle ear prosthesis is done by exploratory surgery, but less invasive procedures are always preferred.

Middle ear prostheses are generally made of polytetrafluoroethylene (PTFE), titanium, or stainless steel, with PTFE preferred because it most closely mimics the natural vibrations of the stapes. However, PTFE is not radiopaque. Thus, should a PTFE prosthesis become dislocated, the only way of determining this dislocation is surgical exploration. Also, even when titanium or stainless steel are used, the images provided by computed tomographic (CT) scan have a halo effect and an accurate location of a prosthesis cannot be determined.

In many applications it is important to see the location of a middle ear prosthesis at an accuracy of fractions of a millimeter. For example, it is useful to determine the depth of penetration of a penetrating prosthesis into the vestibule. However, CT scans of metal prostheses can be over- or under-estimated by as much as 0.5 mm, which is a substantial portion of the overall size of a middle ear prosthesis that are generally available in lengths from 3.5 to 5.25 mm and diameters of 0.4 to 0.8 mm. Additionally, polymer prostheses cannot be accurately measured in the vestibule via CT scans.

It is known to introduce radiopaque markers into various medical devices. For example, U.S. Pat. No. 5,300,048 discloses a flexible plastic material catheter having a distal tubular member portion with a radiopaque agent for radiographic viewing. WO 2005/000165 discloses a stent with a PTFE inner covering, a PTFE outer covering and a radiopaque marker between the inner covering and the outer covering. U.S. Pat. No. 6,635,082 discloses an intraductal medical device having radiopaque marker material deposited on the surface of the medical device to assist visualization of the device during implantation. WO 2006/028370 discloses a hydrogel for an intervertebral disc nucleus comprising at least one monomer comprising iodine or bromine. However, the above devices are either not intended for prolonged use or use in close proximity to the inner ear.

Polytetrafluoroethylene, titanium and stainless steel are well known biocompatible materials that have long been used in middle ear prostheses. However, negative effects are observed when these prostheses unintentionally enter the inner ear. This is because the inner ear is extremely sensitive and prone to deterioration from foreign materials introduced into the body, either in the ear or elsewhere.

Most concerning of the ear's sensitivity to foreign substances is ototoxicity. Ototoxicity is caused by certain therapeutic agents that cause functional impairment and cellular degeneration of the inner ear. The results of exposure to ototoxic therapeutics include temporary and permanent hearing loss. Many different therapeutics have been known to cause ototoxicity, such as aminoglycoside antibiotics (e.g., gentamicin, streptomycin, kanamycin, etc.), anti-neoplastics (e.g., cisplastin), environmental chemicals (e.g., butyl nitirite, mercury, styrene, tin, lead, nickel, manganese, etc.), loop diuretics (e.g., bumetanide, ethacrynic acid, furosemide, etc.), aspirin and quinine products, and cis-platinum. The effects of ototoxicity may be experienced immediately or even months after treatment with an ototoxic therapeutic has ceased. The effects of ototoxicity may be reversible, but sometimes are permanent.

The proximity of an ototoxic therapeutic to the inner ear, and the length of use of an ototoxic therapeutic can influence the likelihood of hearing loss caused by ototoxicity. For example, the use of aminoglycoside antibiotics, a known ototoxic therapeutic, in eardrops has shown an increased likelihood of ototoxicity. Some otolaryngologists believe the proximity of the aminoglycoside eardrops to the round window increases the likelihood of ototoxicity, especially if there is a perforation in the tympanic membrane. In one study, 124 patients suffering from chronic otitis media were given an aminoglycoside (neomycin) for greater than one year and showed a hearing loss of 6 dB on average. Podoshin L, Fradis M, Ben David J. Ototoxicity of ear drops in patients suffering from chronic otitis media. J. Laryngol. Otol. January 1989; 103(1):46-50 As is seen from the numerous causes of ototoxicity, the inner ear is very sensitive to foreign materials. Thus, introducing foreign materials into the body—especially in close proximity to the inner ear—can have negative impacts on a patient's hearing, and great care must be taken when introducing new materials into the middle ear.

Accordingly, there is a need for a middle ear prosthesis that is biocompatible, performs as close as possible to a natural middle ear mechanism and that can be detected post-operation without exploratory surgery. The middle ear prosthesis must also be suitable for prolonged use without affecting the inner ear, such as by causing ototoxicity.

The appropriate components of each of the foregoing may be selected for the present disclosure in embodiments thereof, and the entire disclosure of the above-mentioned and following references are totally incorporated herein by reference.

### SUMMARY

The present disclosure addresses these and other needs, by providing biocompatible middle ear prostheses including a radiopaque marking material, and a method for detecting the location of a biocompatible middle ear prosthesis by exposing a biocompatible middle ear prosthesis including a radiopaque marking material to a radiation source and viewing an image created by the exposure to the radiation source.

A biocompatible middle ear prosthesis comprising:

at least one polymer; and

at least one radiopaque marking material,

wherein at least a portion of the biocompatible middle ear prosthesis is configured to replace all or part of the middle ear ossicles.

In another embodiment, the present disclosure provides a method for detecting the location of a biocompatible middle ear prosthesis comprising:

implanting at least one biocompatible middle ear prosthesis into the middle ear, the biocompatible middle ear prosthesis comprising a polymer and a radiopaque marking material;

exposing the at least one biocompatible middle ear prosthesis to a radiation source;

creating an image from the exposure of the biocompatible middle ear prosthesis to the radiation source; and

viewing the image created from the exposure of the biocompatible middle ear prosthesis to a radiation source to determine the location of the biocompatible middle ear prosthesis in the middle ear.

### BRIEF DESCRIPTION OF THE DRAWINGS

FIG. 1 is an image of the ossicles of a middle ear.

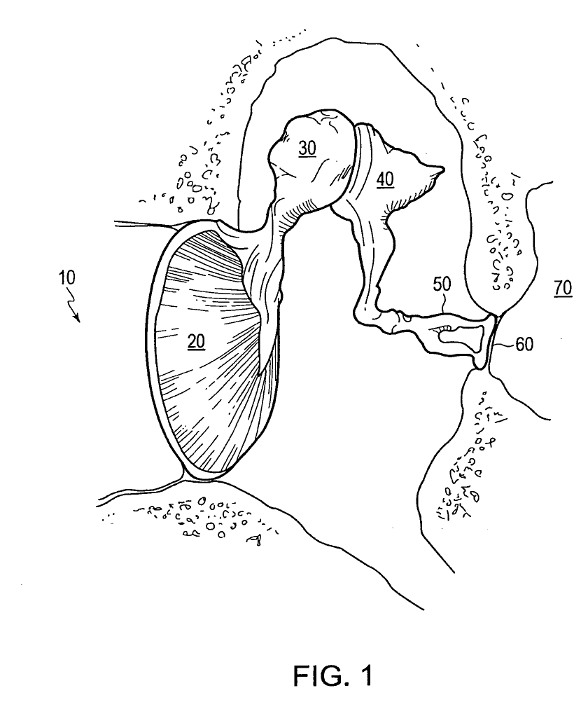

FIG. 2 is an image of an exemplary stapes replacement prosthesis implanted in the middle ear.

### DETAILED DESCRIPTION OF EMBODIMENTS

Embodiments of the disclosure provide biocompatible radiopaque middle ear prostheses. The biocompatible radiopaque middle ear prostheses generally contain fluoropolymers and radiopaque marking materials and are implanted in the middle ear to replace, or restore functionality, to a damaged middle ear mechanism. Another Embodiment of the disclosure provides a method for detecting the location of a biocompatible middle ear prosthesis containing a radiopaque marking material by exposing the biocompatible middle ear prosthesis to a radiation source and viewing an image created by the exposure of the biocompatible middle ear prosthesis to a radiation source to determine the location of the biocompatible middle ear prosthesis.

The structure of a biocompatible middle ear prosthesis according to the disclosure is not limited and may be any structure presently known or devised hereafter that includes a polymer and radiopaque marking materials. For example, the biocompatible prosthesis may be a piston-type prosthesis, such as those disclosed in U.S. Pat. Nos. 3,196,462 and 3,711,869 of which the entire disclosure is hereby totally incorporated by reference. The biocompatible prostheses of this disclosure may also comprise a means for securing the biocompatible prosthesis to the incus, such as the means disclosed in U.S. Pat. Nos. 5,370,689 and 6,197,060 of which the entire disclosure is hereby totally incorporated by reference. The biocompatible prosthesis can also be any total ossicular replacement prosthesis (TORP) or any partial ossicular replacement prosthesis (PORP) known in the art or hereinafter developed, such as the Centered Micron Adjustable TORP+Dornhoffer titanium footplate shoe or the Dornhoffer Malleable PORP (model nos. 7014-3301 and 7014-5842, respectively, both manufactured by Gyrus ACMI, Inc.) or the like.

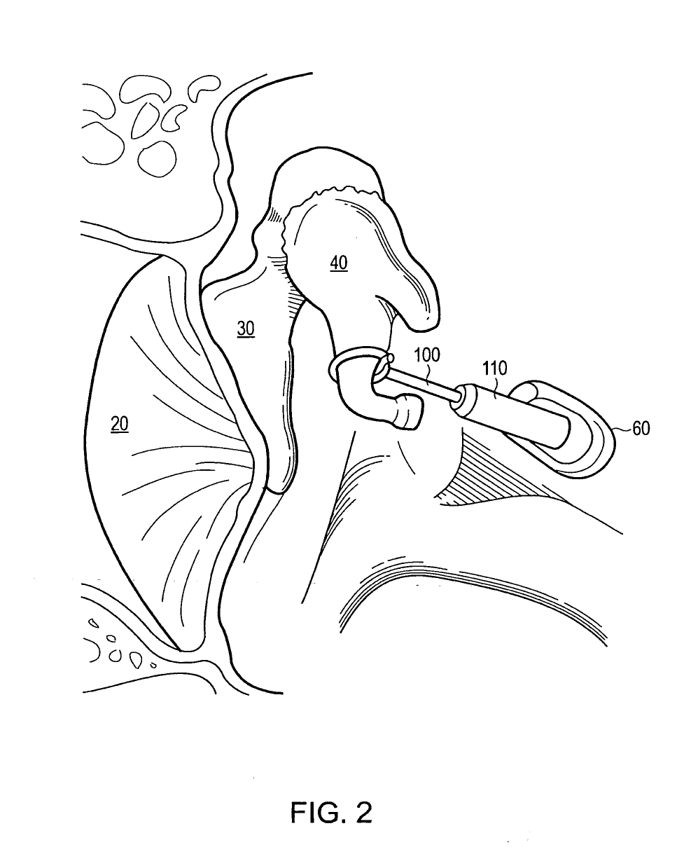

FIG. 2 depicts an exemplary prosthesis. The prosthesis comprises a polymer base 110 that contacts, at its bottom portion, the oval window of the cochlea 60. In embodiments, the polymer base comprises one or more radiopaque marking materials. A hook 100 extends from the top portion of the polymer base 110 of the prosthesis. The hook 100 is securely fastened to, and in direct contact with, the incus 40. The hook 100 may be made of any suitable material. In embodiments, the hook is a metallic hook. In embodiments, the hook comprises titanium, platinum, nitinol, or stainless steel. The hook may be attached to the incus by any known method, for example, by crimping or by applying heat to the hook. In embodiments, heat is applied to the hook via a laser. The exemplary prosthesis depicted in FIG. 2 completely replaces the stapes (not depicted), which would span from the oval window of the cochlea 60 to the nodule of the incus 40, thereby transmitting vibrations from the incus to the oval window and ultimately into the inner ear, as discussed above.

Polymers that may be used in biocompatible middle ear prostheses are not particularly limited. In embodiments, suitable polymers include acrylate and/or methacrylate polymers such as poly(methyl methacrylate), polyetheretherketone, polycarbonates, polyethylenes (PE) (including high-density polyethylene (HDPE), medium-density polyethylene, low-density polyethylene (LDPE), cross-linked high-density polyethylene (XLPE), linear low-density polyethylene (LLDPE), ultra low-density polyethylene, and very low-density polyethylene), oxidized polyethylene, polypropylene (PP), polypropylene copolymer (PPCO), polyisobutylene, polystyrenes, poly(styrene)-co-(ethylene), polysulfones, polyethersulfones, polyarylsulfones, polyarylethers, polyolefins, polyacrylates, polyvinyl derivatives, cellulose derivatives, polyurethanes, polyamides, polyimides, polyesters, silicone resins, epoxy resins, polyvinyl alcohol, polyacrylic acid, polyallomer (PA), polymethylpentene (PMP or TPX), polyketone (PK), polyethylene terephthalates (PET), including polyethylene terephthalate G copolymer (PETG) and oriented PET, polystyrene (PS), polyvinylchloride (PVC), naphthalate, polybutylene terephthalate, thermoplastic elastomer (TPE), mixtures thereof, and the like; engineered resins such as polyamide (such as nylon), polyphenylene oxides (PPO), polysulfone (PSF), mixtures thereof, and the like; mixtures thereof; and the like.

In embodiments, the polymer of the middle ear prosthesis is a fluoropolymer. Suitable fluoropolymers include, but are not limited to, Halar® ethylene-chlorotrifluoroethylene copolymer (ECTFE) (Allied Chemical Corporation, Morristown, N.J.), Tefzel® ethylene-tetrafluoroethylene (ETFE) (E.I. duPont de Nemours and Co. Wilmington, Del.), tetrafluoroethylene (TFE), polytetrafluoroethylene fluorinated ethylene propylene (PTFE-FEP), polytetrafluoroethylene (PTFE), polyethylenefluoride, polytetrafluoroethylene perfluoroalkoxy (PTFE-PFA), and polyvinylidene fluoride (PVDF. In still other embodiments, the fluoropolymer material can be a post-fluorinated polymer material. As used herein, the term “post-fluorinated” refers to any polymer, at least a surface of which is fluorinated subsequent to formation of the polymer material. Thus, for example, the term refers to polymeric materials wherein at least a surface of the polymer material is subsequently fluorinated by suitable treatment methods to introduce fluorine species or atoms into at least the surface layer of the polymeric material. Suitable polymers that can be subjected to post-fluorination treatment include, but are not limited to, polyolefins such as polyethylene (PE) (including high-density polyethylene (HDPE), medium-density polyethylene; low-density polyethylene (LDPE), cross-linked high-density polyethylene (XLPE), linear low-density polyethylene (LLDPE), ultra low-density polyethylene, and very low-density polyethylene), polycarbonate (PC), polypropylene (PP), polypropylene copolymer (PPCO), polyallomer (PA), polymethylpentene (PMP or TPX), polyketone (PK), polyethylene terephthalates (PET), including polyethylene terephthalate G copolymer (PETG) and oriented PET, polystyrene (PS), polyvinylchloride (PVC), naphthalate, polybutylene terephthalate, thermoplastic elastomer (TPE), mixtures thereof, and the like; engineered resins such as polyamide (such as nylon), polyphenylene oxides (PPO), polysulfone (PSF), mixtures thereof, and the like; mixtures thereof; and the like. Particularly suitable fluoropolymers in embodiments are PTFE and polyethylenefluoride. The amount of the fluoropolymer used in the biocompatible prosthesis may be any appropriate amount.

The radiopaque marking material that may be used in biocompatible middle ear prostheses can be any marking material that provides an image when exposed to a radiation source. In embodiments, exemplary radiopaque marking materials include barium, bismuth, tantalum, platinum, stainless steel, titanium, barium compounds such as barium sulfate, organic iodo acids such as iodo carboxylic acids, triiodophenol, iodoform, and tetraiodoethylene. In embodiments, barium and bismuth compounds are particularly useful, as they have been found to meet the above-described needs of maintaining the biocompatibility and performance properties of the implant, while enabling precise visualization of the location of the implant by non-invasion means such as radiation and the like. Furthermore, these radiopaque materials are believed to not contribute to ototoxicity problems, which is a very important consideration when any new foreign materials are introduced into the body, particularly directly into the middle ear area. The amount of the radiopaque marking material used in the biocompatible prostheses may be any amount sufficient to render the biocompatible prosthesis radiopaque. An exemplary amount of radiopaque marking material in embodiments of the biocompatible prostheses may be from about 1% to about 20%, such as from about 5% to about 15%, and from about 7% to about 12%.

Methods for combining polymers and radiopaque marking materials are known, and any method that provides uniform distribution of the radiopaque marking material within the polymer may be used. For example, the radiopaque marking material may be added to the polymer using a twin screw extruder, as disclosed in U.S. Pat. No. 5,300,048. Other methods for combining polymers and radiopaque marking materials that may be used are standard blenders and mixers common throughout the plastics industry. The combined polymer and radiopaque marking material may then be manufactured by known methods and used as all or part of a biocompatible middle ear prosthesis.

The procedure for implanting the biocompatible prostheses of the disclosure may be any procedure presently known or devised hereafter for implanting a prosthesis in the middle ear. For example, the procedure may be a stapedectomy, which replaces the stapes with a prosthesis. To accurately and effectively replace a damaged stapes, a cutting block and stapes measuring device are generally used. The cutting block is used to trim the damaged stapes and/or the prosthesis, and the stapes measuring device is used to accurately measure the stapes and/or the prosthesis. Any cutting block or stapes measuring device known or hereinafter devised may be used, such as those manufactured by Gyrus ACMI, Inc. (such as model nos. 7013-5801 and 269800, respectively). The biocompatible prosthesis of the disclosure may be implanted to replace all of the stapes. By implanting a biocompatible middle ear prosthesis including a radiopaque marking material, the location of the biocompatible middle ear prosthesis can be subsequently determined without exploratory surgery by exposing the biocompatible middle ear prosthesis to a radiation source.

The radiation source used to determine the location of a radiopaque biocompatible middle ear prosthesis is not limited and can be any type of radiation source capable of providing an image showing the location of the biocompatible radiopaque prosthesis in the middle ear. In embodiments, the radiation source is provided by a CT imaging device. The CT device scans a patient's head, thereby exposing the biocompatible middle ear prosthesis to a radiation source. A portion of the radiation source is absorbed by the radiopaque marking material and other portions of the radiation source pass through the body and create an image in which the location of the biocompatible prosthesis is depicted. However, the radiation source may also be provided by an X-ray imaging device, a magnetic resonance imaging device or the like.

A physician may view the image created by the radiation source by any means known in the art. The image depicting the location of the biocompatible middle ear prosthesis including radiopaque marking material may be viewed on a screen, developed on a film or viewed by any other means known now or hereinafter devised. By viewing the image depicting the location of the biocompatible middle ear prosthesis including a radiopaque marking material, a physician can determine if the biocompatible middle ear prosthesis has become dislocated after surgery is complete, or if the biocompatible middle ear prosthesis has settled too far into the vestibule.

It will be appreciated that various of the above-disclosed and other features and functions, or alternatives thereof, may be desirably combined into many other different applications. Also that various presently unforeseen or unanticipated alternatives, modifications, variations or improvements therein may be subsequently made by those skilled in the art which are also intended to be encompassed by the following claims.

What is claimed is:

1. A biocompatible middle ear prosthesis comprising:
at least one polymer; and
at least one radiopaque marking material,
wherein at least a portion of the biocompatible middle ear prosthesis is configured to replace all or part of the middle ear ossicles.

2. The biocompatible middle ear prosthesis according to claim 1, wherein the at least one radiopaque marking material is dispersed in the polymer.

3. The biocompatible middle ear prosthesis according to claim 1, wherein the polymer is chosen from the group consisting of polyfluoroethylene, polytetrafluoroethylene, poly(methyl methacrylate) and polyetheretherketone.

4. The biocompatible middle ear prosthesis according to claim 1, wherein the radiopaque marking material is chosen from the group consisting of barium, bismuth, titanium, and iodo acids.

5. The biocompatible middle ear prosthesis according to claim 1, wherein the radiopaque marking material is barium.

6. The biocompatible middle ear prosthesis according to claim 1, wherein the radiopaque marking material is bismuth.

7. The biocompatible middle ear prosthesis according to claim 1, wherein the at least one radiopaque marking material is present in the biocompatible prosthesis in an amount from 1% to 20% by weight.

8. The biocompatible middle ear prosthesis according to claim 1, further comprising a hook extending from a polymer base.

9. The biocompatible middle ear prosthesis according to claim 6, wherein the hook comprises a material chosen from the group consisting of titanium, stainless steel, nitinol, and platinum.

10. The biocompatible middle ear prosthesis according to claim 6, wherein the hook is configured to be securely fastened to the incus.

11. The biocompatible middle ear prosthesis according to claim 1, wherein the biocompatible prosthesis is implanted to replace the stapes.

12. A saleable kit comprising:
the biocompatible middle ear prosthesis according to claim 1;
a cutting block; and
a stapes measuring device.

13. A medical procedure comprising implanting a biocompatible middle ear prosthesis according to claim 1 into a middle ear of a patient.

14. The medical procedure of claim 13, wherein the patient is a human being.

15. A method for detecting the location of a biocompatible middle ear prosthesis comprising:
implanting at least one biocompatible middle ear prosthesis into the middle ear, the biocompatible middle ear prosthesis comprising a polymer and a radiopaque marking material;
exposing the at least one biocompatible middle ear prosthesis to a radiation source;
creating an image from the exposure of the biocompatible middle ear prosthesis to the radiation source; and
viewing the image created from the exposure of the biocompatible middle ear prosthesis to a radiation source to determine the location of the biocompatible middle ear prosthesis in the middle ear.

16. The method of claim 15, wherein the implanting comprises conducting a stapedectomy.

17. The method of claim 15, wherein the radiation source is provided by a computed tomographic imaging device.
